(19) United States
(12) Patent Application Publication
(10) Pub. No.: US 2015/0306162 A1
(43) Pub. Date: Oct. 29, 2015

(54) USE OF A COMPOSITION COMPRISING ARABIC GUM (AG) FOR IMPROVING GUT IMPERMEABILITY

(71) Applicant: NEXIRA INTERNATIONAL, Rouen (FR)

(72) Inventor: David DAGUET, Leves (FR)

(21) Appl. No.: 14/262,086

(22) Filed: Apr. 25, 2014

(51) Int. Cl. A61K 36/48; A61K 45/06

(52) U.S. Cl. CPC A61K 36/48; A61K 45/06

(57) ABSTRACT

A method for improving gut impermeability including administering to a subject a sufficient amount of a composition comprising acacia gum (AG) and fructo-oligosaccharides (FOS) wherein the composition is in solid form such as tablets, bakery products, confectionary, capsules, powder sachets and boxes or in liquid form.

The present invention relates the use of a composition comprising arabic gum (AG) for improving gut impermeability.

### BACKGROUND OF THE DISCLOSURE

Leaky Gut identifies the association between disrupted intestinal barrier function and the development of autoimmune and inflammatory diseases. The epithelium maintains its selective barrier function through the formation of complex protein-protein networks that mechanically link adjacent cells and seal the intercellular space. An altered transcellular/paracellular equilibrium pathway is involved in the Leaky Gut ethiology. This improper functioning or regulation, involves the tight junctions that seems to be responsible to larger intercellular spaces, at the expense of the transcellular pathway, with luminal element passage through the barrier, with a consecutive local and systemic inflammation.

Leaky Gut is studied nowadays because it is supposed to be involved in some serious health troubles or diseases (Groschwitz et al. 2009—J Allergy Clin Immunol. 124(1):3-20), like chronic fatigue syndrome (Maes et al. 2009—Neuro Endocrinol Lett. 30(3):300-11), Inflammatory Bowel Syndrome (IBS) (Gecse et al. 2012—Digestion. 85(1):40-6), metabolic disorders, inflammatory bowel diseases (Fasano, 2011—Physiol Rev. 91(1):151-75; Camilleri et al. 2012—Neurogastroenterol Motil. 24(6):503-512), type 1 diabetes (Vaarala, 2008-Diabetes. 57:2555-62), allergies, asthma, and autoimmune disease (Fasano, 2012—Clin Rev Allergy Immunol. 42(1):71-8).

Among the possible problems related to a leaky gut, IBS is one of the most common gastrointestinal disorders, afflicting 10 to 15% of the population in developed countries. IBS is considered as a functional trouble because of an apparent absence of findings supporting an organic basis, since there is neither biochemical nor histopathological criteria defined yet.

Some studies suggest that the activated immune system in IBS patients is the result of a raised local antigen exposure associated with an increased permeability of the intestinal epithelial barrier. In fact, it is believed that in IBS, the increased permeability results in an amplified exposure of immune cells to luminal contents.

Biopsy studies revealed persistent increases in the number of mononuclear cells (monocytes/macrophages), T cells and mast cells in patients with post-infectious (PI)-IBS. Besides the higher number in mast cells, there is also an increase in tryptase secretion, which is known to have inflammatory properties, in the colonic lamina propria of patients with IBS. The continued activation of mast cells, even on a very mild basis, could contribute to the motility dysfunction that characterizes IBS, particularly in terms of continued episodes of diarrhea. In addition, mast cells can be found very close to nerve cells in the intestines, perhaps contributing to on-going pain and visceral hypersensitivity that is typical of IBS.

Some patients have increased plasma levels of IL-6 and IL-8 (cytokines primarily produced by monocytes and macrophages) while IL-10 plasma levels were found to be the same. Peripheral blood mononuclear cell (PBMCs) of IBS patients secrete: >IL-6, >IL-113, TNF, >IL-12 and <IL-6: a cytokine profile consistent with a shift towards a T_{H}1 cellular response (adaptive immune system). Moreover, the genotyping of peripheral blood leukocytes of 111 IBS patients and 162 healthy controls showed that the combination of a “high producer” TNF allele and a “low producer” IL-10 allele was more prevalent in patients with IBS.

In conclusion, there are more and more evidences that infection and inflammation are associated with a subset of IBS patients. Researchers have reported that IBS may be caused by bacterial or viral infections in the gastro intestinal (GI) tract. Studies show that people who had gastroenteritis sometimes develop IBS, otherwise called post-infectious (PI)-IBS. The so-called IBS-associated “low-grade inflammation” is, for unknown reasons, present even after that the pathogen has been cleared. This condition may persist for long periods and it has also been detected in patients with IBD in remission. So, it seems that these patients are able to clear the pathogens but not to stop the associated inflammatory response.

Inflammatory Bowel Diseases (IBD) is a group of inflammatory conditions of the colon and small intestine. The major types of IBD are Crohn's disease and ulcerative colitis affecting more than 2.5 million people by the world. IBD can be painful and debilitating. IBD implies that the increased permeability is not simply an epiphenomenon but rather is an important etiological event that causes inflammation in a district distant from where the breach in the intestinal barrier occurs. While a primary defect of the intestinal barrier function may be involved in the early steps of IBD, the production of cytokines as TNF-α, INF-γ, IL-1, IL-4, IL-5, IL-10, IL-12 . . . (Sanchez-Munoz, 2008-World J Gastroenterol 14(27):4280-4288), secondary to the inflammatory process serve to perpetuate the increased intestinal permeability. In this manner, a vicious cycle is created in which barrier dysfunction allows further leakage of luminal contents, thereby triggering an immune response that in turn promotes further leakiness (Fasano, 2011-Ohysiol Rev 91:151-175).

Last but not least, in general, at the molecular level, both IBD and IBS seem to be very similar. They share many common symptoms. Of the things that they share, altered mucosal permeability is characteristic. Additionally, there is an altered interaction of the mucosal flora with immune cell activation.

Accordingly, there is a continuing need for nutritional and therapeutical means of effectively improving the gut impermeability.

### DETAILED DESCRIPTION OF THE PRESENT INVENTION

The present invention is based on the unexpected finding that a composition comprising arabic gum (AG) can be used for improving gut impermeability.

Within the meaning of the present invention arabic gum (AG) is defined as a natural sap that exudes from stems and branches of acacia trees (leguminosae), which grow in the Sahel zone of Africa. The only two botanical species allowed for food applications are Acacia Senegal and Acacia seyal (cf. FAO specification for Acacia gum (1990)). It is a heteropolysaccharide of high molecular weight (around 200 kDa), characterized by a ratio of sugar composition of galactose/arabinose ≦0.9 and another ratio of arabinose/rhamnose ≧10 (Menzies et al., 1996).

According to the present invention “improve the gut impermability” means that the impermeability of the gut of a human—with symptoms of leaky gut treated with the composition of the invention—is statistically different when measured by tests (see below) from the gut impermeability of a control individual (i.e. it is statistically above the impermeability if the control individual).

The control individual is a person presenting the symptoms of leaky gut that is not treated with the composition of the invention comprising AG (but may receive another composition).

The improvement of the gut impermeability encompasses the complete restoration of the gut impermeability (i.e. the gut impermeability being then statistically identical to a human that does not present the symptoms of leaky gut).

Intestinal permeability is the phenomenon of the gut wall exhibiting permeability. It is a normal function of the intestine to exhibit some permeability, but to maintain a barrier function whereby potentially harmful functions are prevented from leaving the intestine and migrating to the body more widely. In a healthy human intestine small particles (<4 Å in radius) can migrate through tight junction pores.

In order to measure the effect of AG in improving the impermeability of the gastrointestinal tract tests can be performed such as:
Trans Epithelial Electric Resistance (TEER) measurements as an indication of the enterocyte monolayer membrane integrity and decreased permeability,
Evaluation of the Lucyfer yellow permeation in the BL compartment as an indication of the monolayer permeability,
Measurement of cytokines production in the BL compartment (IL-8, IL-6, TGF-β, IL-10) and NF-κB activity following the contact with the SHIME suspension.

The improvement of the impermeability may be defined when referring to the TEER measurement as an increase of the TEER of at least 35% when compared to the control, preferably of at least 40% and even more preferably of at least 50%.

The present invention therefore relates to a pharmaceutical composition contains AG as the active ingredient.

The pharmaceutical composition according to the invention allows the improvement of gut impermability in human beings.

The pharmaceutical composition according to the present invention contains the active ingredient in sufficient amount to provide the desired therapeutic effect, i.e., improving the gut impermeablility

Preferably, the following amounts of active ingredient are used to prepare the pharmaceutical composition according to the invention: from 1 to 60 g, preferably from 5 to 30 g of AG.

According to a further aspect, the pharmaceutical composition of the invention further comprises fructo-oligosaccharides (FOS). In an even more specific aspect the FOS are present in a amount of 1 to 50% percent of the weight of the composition

Fructooligosaccharides (FOS) refer to short-chain oligosaccharides comprised of D-fructose and D-glucose, containing from three to five monosaccharide units. FOS, also called neosugar and short-chain FOS, are produced on a commercial scale from sucrose using a fungal fructosyltransferase enzyme. FOS are resistant to digestion in the upper gastrointestinal tract. They act to stimulate the growth of Bifidobacterium species in the large intestine and contribute to the restoration of the intestinal impermeability and have an anti-inflammatory effect.

The pharmaceutical composition according to the present invention can be formulated in any galenical form necessary to its administration. In particular, as regards oral administration, the compositions according to the invention can be formulated in the form of coated or uncoated, effervescent, soluble, orodispersible, gastroresistant or modified-release tablets; of sugar-coated tablets; of hard-shell capsules (or gelatine capsules); of soft-shell capsules; of small pills; of granules; of pills; or of lozenges. The pharmaceutical compositions according to the present invention may therefore comprise, in addition to the active ingredients known to the skilled artisan and which is necessary for the preparation of the pharmaceutical composition in the desired form.

Thus, the pharmaceutical composition according to the present invention can be formulated in a liquid or solid forms such as powders, bars, supplements and any other food product as known in the art. The powder can be reconstituted to form a liquid. The composition may include at least amino acids like L-glutamine, non-fermentescible carbohydrates, vitamins like vitamin D, polyphenols like quercetin, plant extracts like turmeric, aloe vera, plantain, calendula, essential fatty acids like linoleic acid, alpha-linolenic acid, probiotics like Lactobacillus and Acidophilus sp., minerals like zinc, enzymes like SOD, pepsin or pancreatin.

The term “liquid” as used herein, unless otherwise specified, refers to products in ready-to-drink liquid form, concentrated form, and liquids made by reconstituting the powders described herein prior to use.

These liquids are most typically formulated as suspensions, emulsions or clear or substantially clear liquids.

Emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The emulsions may be and typically are shelf stable. The emulsions typically contain up to about 95% by weight of water, including from about 50% to about 95%, also including from about 60% to about 90%, and also including from about 70% to about 85%, of water by weight of the emulsions. The emulsions may have a variety of product densities, but most typically have a density greater than about 1.03 g/ml, including greater than about 1.04 g/ml, including greater than about 1.055 g/ml, including from about 1.06 g/ml to about 1.12 g/ml, and also including from about 1.085 g/ml to about 1.10 g/ml.

The emulsion may have a pH ranging from about 3.5 to about 8, but are most advantageously in a range of from about 4.5 to about 7.5, including from about 5.5 to about 7.3, including from about 6.2 to about 7.

The solids may be in any form, including bars, tablets, and the like, but are typically in the form of flowable or substantially flowable particulate formulations, or at least particulate formulations. Particularly suitable solid product forms include spray dried, agglomerated or dryblended powder compositions. The formulations can easily be scooped and measured with a spoon or similar other device, wherein the formulations can easily be reconstituted by the intended user with a suitable aqueous liquid, typically water, to form a formulation for immediate oral or enteral use. In this context, “immediate” use generally means within about 48 hours, most typically within about 24 hours, preferably right after reconstitution.

The term “powder” as used herein, unless otherwise specified, refers to formulations in flowable or scoopable form that can be reconstituted with water or another aqueous liquid prior to consumption and includes both spray dried and dry mixed/dry blended powders.

The pharmaceutical composition of the invention can be administrated to any patient who is suffering from gut permeability.

Thus the present invention also related to a pharmaceutical composition as defined above for retoring gut impermeability in a human being.

Preferably, the present invention relates to a pharmaceutical composition as defined above for the treatment of a subject suffering from gut permeability. Thus, the present invention related to a pharmaceutical composition for the treatment of abdominal pain, chronic or not, insomnia, bloating, flatulence, shortness of breath, gluten intolerance, malnutrition, muscle cramps, multiple chemical sensitivities, muscle pain, myofascial pain, mood swings, poor exercise tolerance, poor immunity, poor memory, recurrent skin rashes, brittle nails, hair loss, food allergies, constipation, diarrhea, liver dysfunction, brain fatigue, abdominal spasms, constant hunger pains, depleted appetite, Irritable Bowel Syndrome, chemotherapy, food allergies, acne, liver dysfunction or inflammation of the bowel.

The pharmaceutical composition may be administrated at any moment during the day, before, during or after meals, without his having any influence on the efficacy of the treatment. Thus, the pharmaceutical composition of the invention may be administrated one to three times a day continuously during the year, during a period of 1 to 25 weeks, or more preferably of 3 to 17 weeks.

The invention also relates to the use of a pharmaceutical composition as defined above for the preparation of a medicament useful for improving the gut impermability and preferably for the treatment of abdominal pain, chronic or not, insomnia, bloating, flatulence, shortness of breath, gluten intolerance, malnutrition, muscle cramps, multiple chemical sensitivities, muscle pain, mood swings, poor exercise tolerance, poor immunity, poor memory, recurrent skin rashes, brittle nails, hair loss, food allergies, constipation, diarrhea, liver dysfunction, brain fatigue, abdominal spasms, constant hunger pains, depleted appetite, Irritable Bowel Syndrome, chemotherapy, food allergies, acne, liver dysfunction or inflammation of the bowel.

The present invention also related to a method for improving gut impermeability comprising administering to a subject a sufficient amount of a pharmaceutical composition as defined above. Preferably, the present invention relates to a method for the treatment of abdominal pain, chronic or not, insomnia, bloating, flatulence, shortness of breath, gluten intolerance, malnutrition, muscle cramps, multiple chemical sensitivities, muscle pain, myofascial pain, mood swings, poor exercise tolerance, poor immunity, poor memory, recurrent skin rashes, brittle nails, hair loss, food allergies, constipation, diarrhea, liver dysfunction, brain fatigue, abdominal spasms, constant hunger pains, depleted appetite, Irritable Bowel Syndrome, chemotherapy, food allergies, acne, liver dysfunction or inflammation of the bowel or of anaphylactoid reactions, fevers of unknown origin, anxiety, hemorrhoids, heartburn, migraines, mood swings, poor exercise tolerance, recurrent bladder infections, recurrent vaginal infections, swollen lympth glands, anal irritation, depression, sluggishness, celiac disease, multiple Sclerosis, autism, fibromyalgia, Chronic Fatigue Syndrome, eczema, dermatitis, ulcerative colitis, candidiasis, chronic hepatitis, asthma, cystic fibrosis, accelerated aging, endotoxemia, colon cancer, Crohn's disease, giardia, arthritis, pancreatic dysfunction, Hives, alcoholism, anyklosing spondylitis, inflammatory bowel disease, liver dysfunction, psoriasis, schizophrenia, alcoholism.

The present invention also relates to a method for improving the gut impermeability by administration of a composition, as defined above, one to three times a day continuously during the year, during a period of 1 to 25 weeks, or more preferably of 3 to 17 weeks.

According to another aspect of the invention, the composition further comprises fructo-oligosaccharides (FOS). These two active ingredients can be administrated in the form of a unitary pharmaceutical composition comprising the two active ingredients allowing administration of said composition to the patient in a single intake.

Nevertheless, a separate administration of the AG and the FOS can also be envisaged. Thus the present invention also relates to a pharmaceutical product containing AG and FOS as a combination (or pharmaceutical kit) for simultaneous, separate or sequential administration.

Preferably, the pharmaceutical product according to the invention is used in the treatment of gut permeability and more particularly for the treatment of abdominal pain, chronic or not, insomnia, bloating, flatulence, shortness of breath, gluten intolerance, malnutrition, muscle cramps, multiple chemical sensitivities, muscle pain, myofascial pain, mood swings, poor exercise tolerance, poor immunity, poor memory, recurrent skin rashes, brittle nails, hair loss, food allergies, constipation, diarrhea, liver dysfunction, brain fatigue, abdominal spasms, constant hunger pains, depleted appetite, Irritable Bowel Syndrome, chemotherapy, food allergies, acne, liver dysfunction or inflammation of the bowel or to treat anaphylactoid reactions, fevers of unknown origin, anxiety, hemorrhoids, heartburn, migraines, mood swings, poor exercise tolerance, recurrent bladder infections, recurrent vaginal infections, swollen lympth glands, anal irritation, depression, sluggishness, celiac disease, multiple Sclerosis, autism, fibromyalgia, Chronic Fatigue Syndrome, eczema, dermatitis, ulcerative colitis, candidiasis, chronic hepatitis, asthma, cystic fibrosis, accelerated aging, endotoxemia, colon cancer, Crohn's disease, giardia, arthritis, pancreatic dysfunction, Hives, alcoholism, anyklosing spondylitis, inflammatory bowel disease, liver dysfunction, psoriasis, schizophrenia, alcoholism.

Preferably, the following daily amounts of active ingredient are used to prepare the pharmaceutical composition according to the invention:
from 1 to 60 g, preferably from 5 to 30 g of AG,
from 1 to 60 g, preferably from 5 to 30 g of FOS.

The pharmaceutical product according to the invention can of course be administrated according to the administration schemes previously defined.

The SHIME renders that possible to evaluate the effect induced by the AG and its metabolites which are produced by the gut microbiota during the digestive steps (and not the pure product alone).

### The SHIME Technology

The study of effect of the AG on the gut impermeability is made using the SHIME, an in vitro continuous model, which allows culturing the complex intestinal microbial ecosystem over a long period and under representative conditions. Moreover, the SHIME allows simulating repeated ingestion of the test product. In fact, according to previous data, AG is mainly fermented in the distal colon (Transverse Colon (TC) and Distal Colon (DC)) and repeated doses of the product are necessary to show an effect on the gut microbial community composition and activity.

The reactor setup was adapted from the SHIME (FIG. 1), representing the gastrointestinal tract of the adult human, as described by Molly et al. (1993—Applied Microbiology and Biotechnology 39(2): 254-258).

The SHIME consists of a succession of five reactors simulating the different parts of the human gastrointestinal tract. The first two reactors are of the fill-and-draw principle to simulate different steps in food uptake and digestion, with peristaltic pumps adding a defined amount of SHIME feed (140 mL 3×/day) and pancreatic and bile liquid (60 mL 3×/day), respectively to the stomach (V1) and duodenum (V2) compartment and emptying the respective reactors after specified intervals. The last three compartments are continuously stirred reactors with constant volume and pH control. Retention time and pH of the different vessels are chosen in order to resemble in vivo conditions in the different parts of the gastrointestinal tract. The overall residence time of the last three vessels, simulating the large intestine, is 72 h. Upon inoculation with fecal microbiota, these reactors simulate the ascending (V3), transverse (V4) and descending (V5) colon. Inoculum preparation, retention time, pH, temperature settings and reactor feed composition were previously described by Possemiers et al. (2004—FEMS Microbiology Ecology 49: 495-507).

The SHIME has been extensively used for more than 15 years for both scientific and industrial projects and has been validated with in vivo parameters. Upon stabilization of the microbial community in the different regions of the colon, a representative microbial community is established in the three colon compartments, which differs both in composition and functionally in the different colon regions.

The human intestinal tract harbours a large and complex community of microbes, which is involved in maintaining human health by preventing colonization by pathogens and by producing nutrients. Microorganisms are not randomly distributed throughout the intestine and those adhering to the gut wall play an important role as a ‘barrier’ against pathogens, instructing mucosal immune responses and occupying a niche at the expense of potentially harmful colonizers.

However, available in vitro strategies did not allow culturing the fraction of microorganisms which adhere to the gut mucosa and were limited to modelling of the luminal microbial community. This means that an important part of the gut ecosystem was not taken into account.

To overcome this problem, ProDigest recently developed an adaptation of the SHIME® which takes into account the colonization of the mucus layer. Being unique in its field, the so-called M-SHIME® allows culturing both the luminal and mucus-associated microbial community.

The gut wall is normally covered with a mucus layer and part of the gut microbial community specifically adapted to live in this specific niche. This means that some microorganisms can preferentially grow when adhering to the mucin surface. The structure and composition of this ecosystem reflects a natural selection at both microbial and host levels, which promote a mutual cooperation in the search of a functional stability. This fraction of bacteria is normally believed to have a key effect in relation to the host's health, due to the direct contact with the host itself. The M-SHIME is a conventional SHIME system with the additional simulation of a gut surface (i.e. plastic beads covered with a mucin agar layer; 50% of them are replaced every 48 hours thus providing a constant surface for bacteria adhesion). This provides a more ecologically-relevant gut microbial community, increasing the survival in the system of those species (e.g. lactobacilli) that otherwise would be quickly washed out. Inclusion of the mucosa compartment increases the value and modeling capacity of the SHIME®.

The M-SHIME has been already validated to simulate the microbial processes occurring in the GIT of UC patients (Vermeiren et al. 2012—FEMS Microbiol Ecol. 79(3): 685-96). As stated by the authors, the use of the M-SHIME with the fecal microbiota from healthy volunteers and UC patients showed that the diversity of the C. coccoides/E. rectale and C. leptum group (butyrate producers), the abundance of F. prausnitzii and the functional gene butyryl-CoA:acetate CoA transferase are decreased in the luminal fractions from UC patients. Moreover, the abundance of Roseburia spp. and butyryl-CoA:acetate CoA transferase was lower also in the mucosal fractions from the UC patients. The results obtained with this model confirmed previous in vitro and in vivo studies (Swidsinski et al., 2005—J Clin Microbiol 43: 3380-3389; Sokol et al., 2006—Inflamm Bowel Dis 12: 106-111; 2009—Inflamm Bowel Dis 15: 1183-1189; Andoh et al., 2011—J Gastroenterol 46: 479-486).

As compared to the regular SHIME, this experiment was shorter considering that, when inoculating the SHIME with a fecal sample from a diseased person, it may not be possible to maintain the ‘diseased’ microbiota for long time. In fact, in absence of the selective pressure of the host, the microbiota tends to a balanced composition.

Samples collected from the different colonic areas of the SHIME systems (both the regular SHIME and the M-SHIME) have been used to evaluate the effect of AG on inflammation and leakiness of the gut.

### Trans Epithelial Electric Resistance (TEER) Measurements and Evaluation of the Lucyfer Yellow Permeation

In order to measure the effect of AG in improving the impermeability of the gastrointestinal tract use was made of the co-culture model shown in FIG. 2, based on the model described by Satsu, H. et al., (2006—Experimental Cell Research, 312: 3909-19).

To set up the system, Caco-2 cells are grown in semi-permeable inserts until enterocyte-like maturation. After 14 days a functional polarized monolayer is formed and the inserts are then placed on top of activated THP-1-macrophages. The presence of THP1 induces damage on the Caco-2 epithelia, thereby affecting barrier integrity (decrease in TEER). Finally, LPS is added on the basolateral (BL) side to induce inflammation (increase in pro-inflammatory cytokine levels).

This IBD-like model can therefore be used for testing the effect of substances that can protect intestinal epithelial barrier integrity (by inducing an increase in TEER) and can reduce the inflammation (by reducing pro-inflammatory cytokines and increasing anti-inflammatory cytokines).

Samples collected from the different compartments of the SHIME have been brought in contact with a monolayer of Caco-2 cells to evaluate the effect of the test product and its metabolites on gut permeability. This effect is normally evaluated at level of the tight junctions. The latter are proteins that keep adjacent epithelial cells together, thereby forming a virtually impermeable barrier to fluids. The Trans-epithelial electrical resistance (TEER) allows measuring the “tightness” of these structures, with high TEER corresponding to a tighter barrier. When damage occurs, these proteins are altered and barrier function is lost. In this case, the TEER is reduced and paracellular transport (between cells) of fluids may increase (FIG. 3). Moreover, the effect on the gut barrier permeability can be observed by analysing the paracellular transport of lucifer yellow (LY).

### Measurement of Pro-Anti-Inflammatory Activity of the Test Products

Chemical, mechanical or pathogen-triggered barrier disruption may lead to influx of bacteria from the lumen into the lamina propria. This activates the immune system, which switches from a physiological “tolerogenic” inflammation into a detrimental pathological inflammation.

An inflammatory signalling cascade will initiate with the production of alarm molecules such as pro-inflammatory cytokines (e.g. IL-8, TNF-α, IL-6) and acute phase proteins (APP). These molecules, among which IL-8 and TNF-α, will induce the recruitment of neutrophils and monocytes to the site of inflammation (FIG. 4). These cells are necessary to kill the bacteria and plug possible breaches in the epithelial wall; however they may also cause tissue disruption.

In a healthy person, the immune activation is counteracted by anti-inflammatory cytokines, such as IL-10 and IL-6 (the last one has a dual role as it can be both pro- and anti-inflammatory). More specifically, IL-10 is able to suppress several cells from both innate and adaptive immune systems, to induce activation of anti-inflammatory molecules and to enhance T regulatory cell function (able to restore immune homeostasis); IL-6 is able to promote death of neutrophils and to inhibit production of pro-inflammatory cytokines by inducing for instance the production of IL1-RA.

In terms of inflammation, TNF-α is one of the most important and dangerous cytokines produced by the immune system as it is able to amplify inflammation (FIG. 4).

When not counteracted, TNF-α can lead to chronic inflammation and even death in cases of acute inflammation. For this reason, anti-TNF-α therapy is widely used in several chronic inflammatory conditions such as rheumatoid arthritis, ankylosing spondylitis, inflammatory bowel disease (IBD) and psoriasis. In IBD for example, anti-TNF-α therapy is commonly used to treat chronic inflammation. However, these have several side effects: long term loss-of-response, higher susceptibility to infections and higher incidence of malignancy (as TNF-α is an anti-tumor molecule).

### BRIEF DESCRIPTION OF THE FIGURES

FIG. 1: Standard setup of the Simulator of the Human Intestinal Microbial Ecosystem (SHIME), consisting of 5 sequential reactors which simulate the different regions of the human intestinal tract.

FIG. 2: co-culture system of Caco-2 cells and THP1 macrophages composed of an apical (AP) and basolateral (BL) side

FIG. 3: scheme of the TEER functionality FIG. 4: TNF-α cascade of inflammation FIG. 5: TEER and LY permeability. at the end of treatment week 1 (T1) and end of treatment week 2 (T2); in AC, TC and DC.

FIG. 6: TEER data. In panel A, ‘Control’ means control period (2 days of starch) and which was set to 100%. In panels A and B, (C) stands for starch control (4 days of treatment).

FIG. 7: Lucyfer yellow permeation. In both panels A and B, (*) represent significantly different from control. In panel A, ‘Control’ means control period (2 days of starch) and which was set to 100%. In panel A and B, (C) stands for starch control (4 days of treatment

FIG. 8: net activity of NF-kβ before and after the addition of LPS in the AC, TC and DC of the SHIMEs treated with AG. (T1) represents the results at the end of treatment week 1, (T2) represents the results at the end of treatment week 2.

FIG. 9: net % concentration of TNF-α and IL-8 in the AC, TC and DC of the SHIMEs treated with AG. (T1) represents the results at the end of treatment week 1, (T2) represents the results at the end of treatment week 2.

FIG. 10: net % concentration of IL-6 and IL-10 in the AC, TC and DC of the SHIMEs treated with AG. (T1) represents the results at the end of treatment week 1, (T2) represents the results at the end of treatment week 2.

FIG. 11: net activity of NF-kB before and after the addition of LPS in the PC and DC of the SHIMEs treated with AG and the control SHIME. In panel A, ‘Control’ means control period (2 days of starch) and which was set to 100%. In panels A and B, (C) stands for starch control (4 days of treatment).

FIG. 12: net % concentration of TNF-α in the PC and DC of the SHIMEs treated with AG and the control SHIME. In panel A, ‘Control’ means control period (2 days of starch) and which was set to 100%. In panels A and B, (C) stands for starch control (4 days of treatment).

FIG. 13: net % concentration of IL-8 in the PC and DC of the SHIMEs treated with AG and the control SHIME. In panel A, ‘Control’ (depicted in red) means control period (2 days of starch) and which was set to 100%. In panels A and B, (C) stands for starch control (4 days of treatment).

FIG. 14: net % concentration of IL-6 in the PC and DC of the SHIMEs treated with AG and the control SHIME. In panel A, ‘Control’ means control period (2 days of starch) and which was set to 100%. In panels A and B, (C) stands for starch control (4 days of treatment).

FIG. 15: net % concentration of IL-10 in the PC and DC of the SHIMEs treated with FOS, AG and the control SHIME. In panel A, ‘Control’ means control period (2 days of starch) and which was set to 100%. In panels A and B, (C) stands for starch control (4 days of treatment).

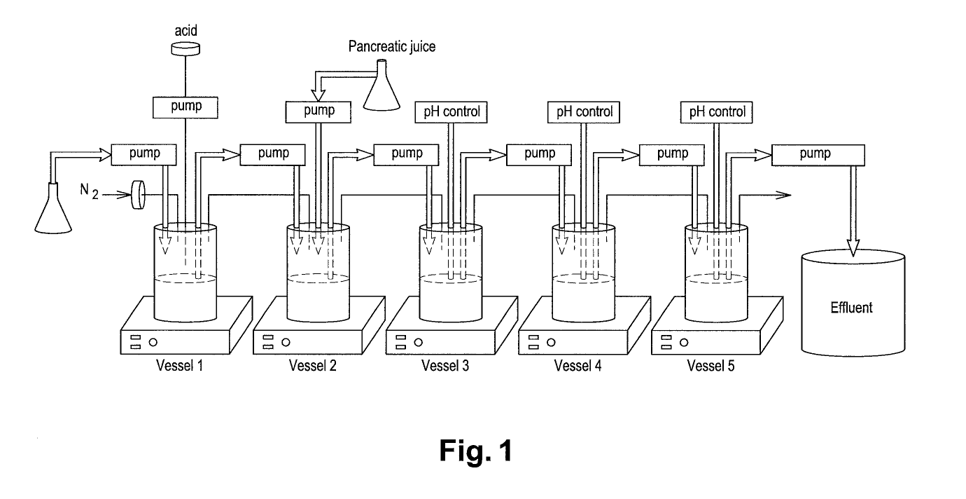

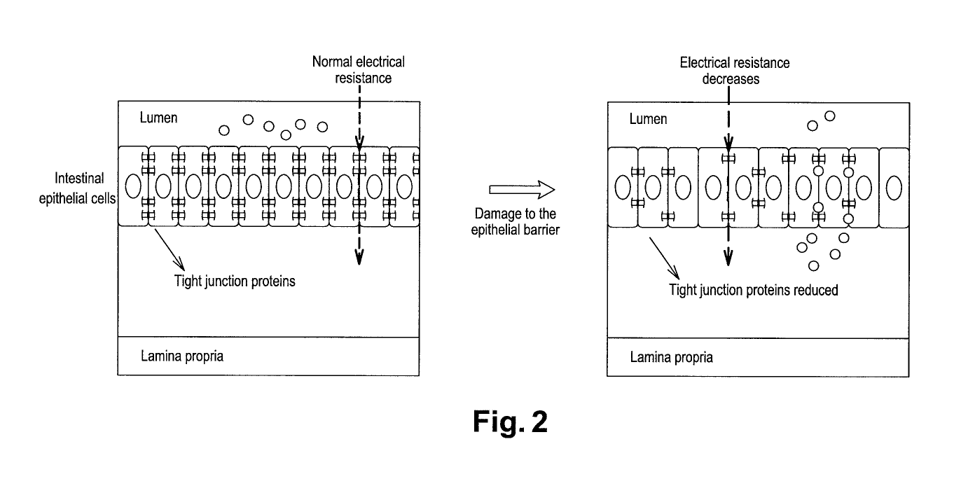

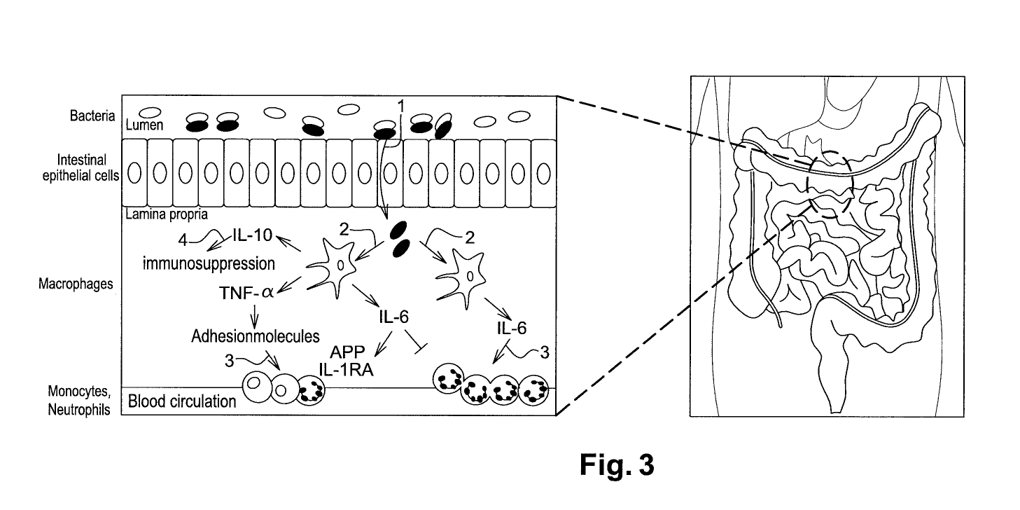

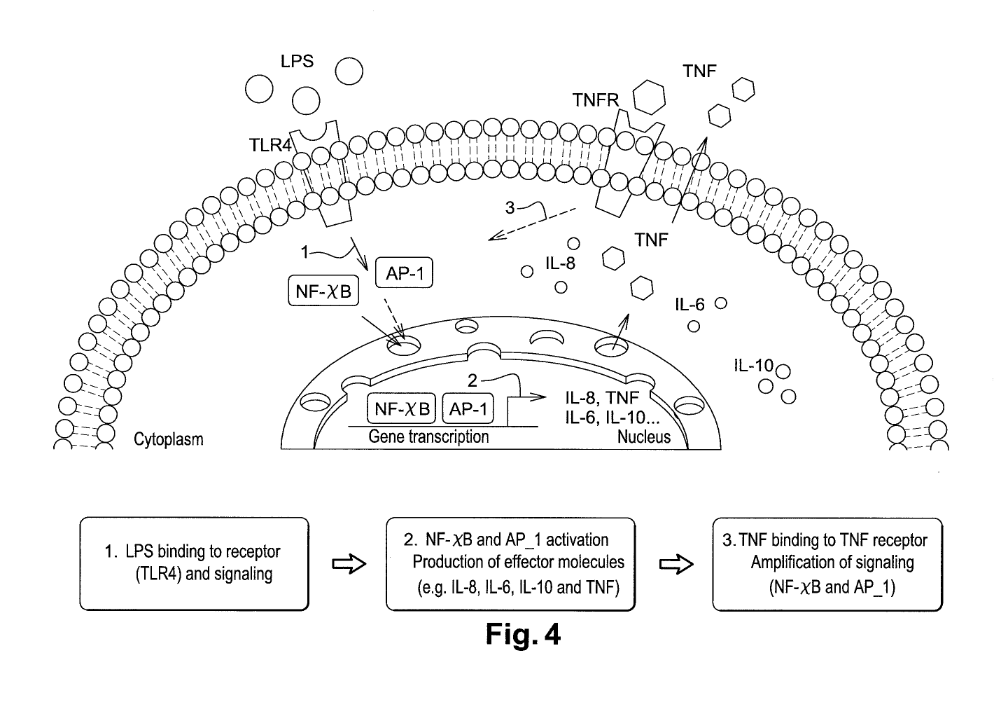

The following non-limiting examples are provided to further illustrate the present invention.

### EXAMPLE 1

### Measure of TEER and LY Paracellular Transport with Samples from the IBS-SHIME

The SHIME experiment was done as follows:
Start up: After inoculation of the colon reactors with an appropriate fecal sample (mild IBS donor), a two-week start up period allowed the microbial community to differentiate in the different reactors depending on the local environmental conditions.
Control period (1 week): This was the actual start of the experiment, in which standard SHIME feed have been dosed to the model for a period of 7 days. The standard medium was composed as follows: Arabinogalactan (1 g/L), Pectin (2 g/L), Xylan (1 g/L), Starch (4.2 g/L), Glucose (0.4 g/L), Yeast extract (3 g/L), Peptone (1 g/L), Mucin (4 g/L), Cysteine (0.5 g/L). Analysis of samples in this period allowed determining the baseline microbial community composition and activity in the different reactors, which have been used as control to compare with the results from the treatment.
Treatment period (2 weeks): In this 2-week period, the SHIME reactor was operated under nominal conditions, but with a modified diet containing a lower amount of starch in the medium compared to that of the basal period. In parallel, the diet of the SHIME has been supplemented with AG or FOS. The dosage rate of the product was 5 g/day. Samples were collected from the colon compartments of the SHIME reactor that was fed with daily doses of AG of 5 g. Samples collected at the end of treatment first week correspond to T1, those collected at the end of the second treatment week correspond to T2.

Samples collected from the different compartments of the IBS-SHIME (AC: Ascending Colon; TC: Transverse Colon; DC: Descending Colon) have been brought in contact with a monolayer of Caco-2 cells (200 μl) to evaluate the effect of the test product and its metabolites on gut permeability. This effect is normally evaluated at level of the tight junctions.

The obtained data have been treated as follows: data have been normalized to the control period, thereby taking into account (and eliminating) the differences already existing before the treatment. Then, the net result was calculated, by taking into account the sequential inter-dependence between colon compartments (AC to TC to DC). Results are shown in FIG. 5.

Although AG shows no protection in the AC when compared to the control treatment (0% and 4% for T1 and T2, respectively), gradually, a protective effect increases towards the distal colon and in the DC, AG is able to have a protective effect on barrier integrity by showing an increase in TEER of nearly 40% (T1) and 50% (T2).

Data from the paracellular transport of LY (graph B) shows an increase of the paracellular transport of LY in the AC. In the TC, the LY transport decreases: 10% (T2) for AG. AG was able to reduce LY transport of 34% in the second week of treatment.

### EXAMPLE 2

### Measure of TEER and LY Paracellular Transport with Samples Collected from the IBD-M-SHIME

The models used to assess the effect of the samples collected from the IBD-M-SHIME are the same described for the IBS-SHIME.

The M-SHIME experiment was done as follows:
Start up: After inoculation of the colon reactors with an appropriate fecal sample (IBD donor), the microbiota was allowed to stabilize in the reactor for 2 days.
Treatment period (4 days): In this 4-day period, the SHIME reactor was operated under nominal conditions for the control reactors) or with a modified diet normally containing a lower amount of starch in the medium and the addition of AG. The dosage rate for both products was 5 g/day. Samples were collected from the colon compartments of the SHIME reactor that was fed with daily doses of AG of 5 g. The samples brought in contact with a monolayer of Caco-2 cells are of 200 μl.

Data are presented as follows: A first set of graphs (always depicted as A) is shown, where the results are normalized to the control period (which consisted of two days of 4 g/L starch). In this way one takes into account (and eliminates) the differences already existing before treatment. Then, a second set of graphs is shown (always depicted as B) where the net result was calculated, by taking into account the sequential inter-dependence between colon compartments (proximal colon→distal colon) [as shown with the IBS data]. Note that in this SHIME, an actual control (4 g/L) of starch was also done during the entire course of the experiment (2 days of control+4 days of treatment). Therefore, this group is also shown.

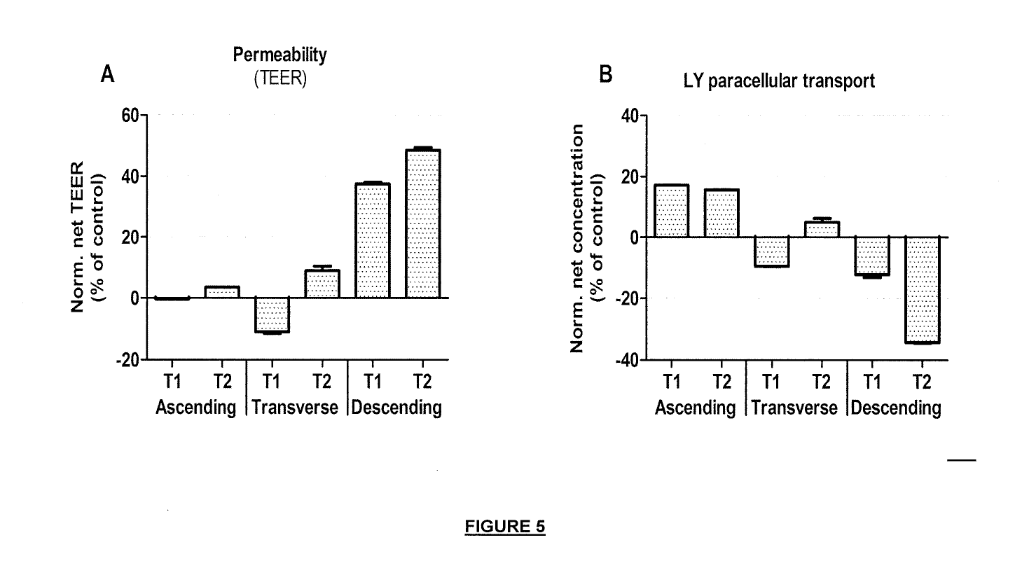

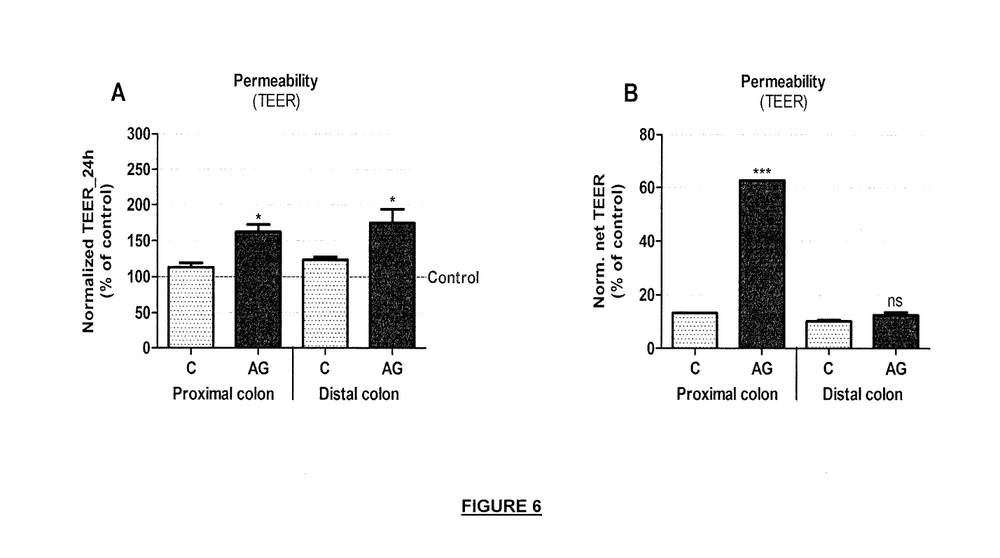

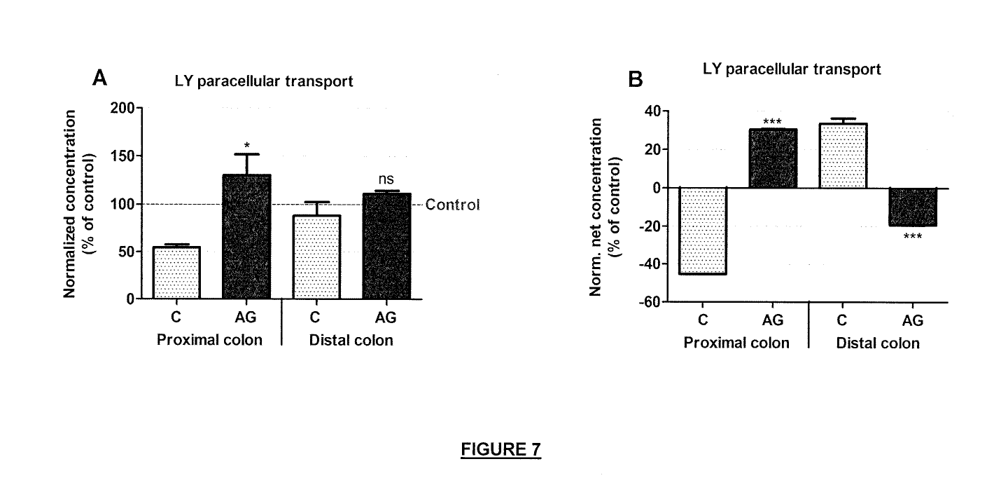

Results are shown in FIGS. 6 and 7.

When considering the TEER parameter, the protective effect of the starch control (C) is rather marginal in both colon compartments: +13% in the proximal colon and +24% in the distal colon when compared to the control period (FIG. 6 A)

However, AG, was able to protect the integrity of the Caco-2 monolayer. This effect was very pronounced in the proximal colon for AG: 63% more protection when compared to the control period (FIG. 6 A), and 50% more when compared to the starch control after 4 days of fermentation (FIG. 6 B).

When taking into account the net result, it is possible to observe that the protective effect was more pronounced in the proximal colon for both fibers. Nevertheless, in the distal colon, AG significantly improved the gut barrier permeability.

Although AG increased the TEER, suggesting a protective effect at the level of the tight junctions, the permeability to small molecules, such as LY, was increased in the proximal colon when compared to the control period (+31%; FIG. 7 A) and to the starch control after 4 days of fermentation (+76%; FIG. 7 B). The same was observed in the distal colon for AG: +11% when compared to the control period and +23% when compared to the starch control (FIG. 7 A).

It is worth noticing that actually, despite these results that appears to be in contrast with those shown in FIG. 6, only a very small amount of LY was detected on the basolateral side for AG: only 4% of the LY originally added was detected on the basolateral compartment of an empty well. This means that in absolute values, the permeability to LY was almost null for both fibers.

When calculating the net result (FIG. 7 B), it is possible to observe that the permeability to LY decreased for AG from the proximal to the distal colon (−20%),

### EXAMPLE 3

### Pro-Anti-Inflammatory Activity of the Test Products

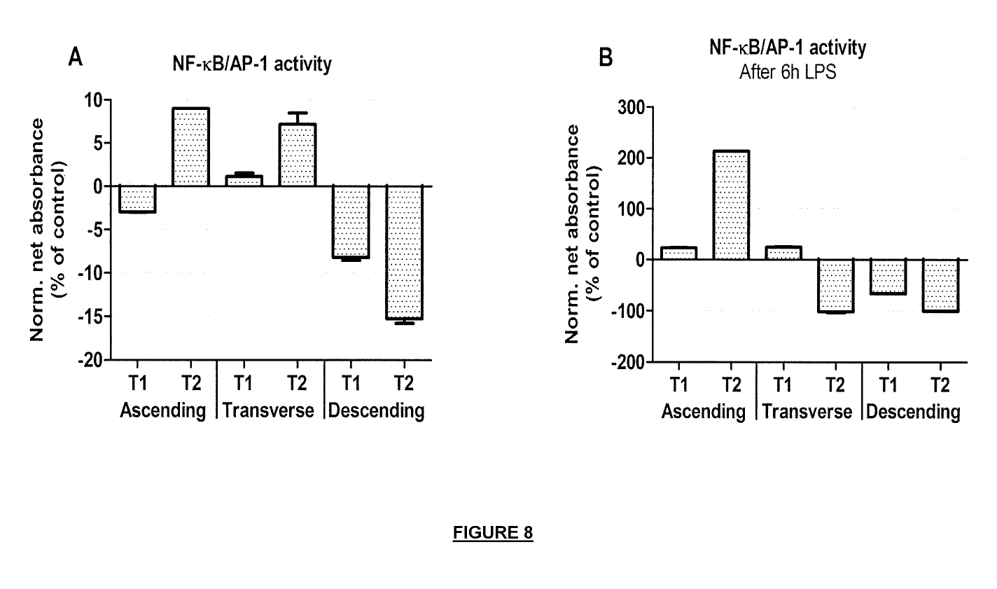

FIGS. 8-10 show the results of the effect of AG on pro-anti-inflammatory cytokines.

The net activity of NF-kB before and after the addition of LPS (100 ng/ml) in the different parts of the colon (AC, TC and DC) of the SHIME treated with AG (cf. Example 1) was measured (FIG. 8).

NF-κB and AP-1 are two of the most important transcription factors involved in immune functions and cellular activity; they are able to induce both pro- and anti-inflammatory molecules and to modulate cell survival and proliferation. These two transcription factors are dramatically induced by lipopolysaccharides (LPS) (isolated from gram negative bacteria).

In absence of a strong inflammation (no addition of LPS) (FIG. 8 A), it was possible to observe that NF-κB/AP-1 activity was enhanced in the TC: +1% (T1) and +7% (T2) for AG while the same activity decreased remarkably in the DC: −8% (T1) and −15% (T2) for AG.

However, after LPS stimulation (FIG. 8 B), AG—although showing an initial increase in the AC—was able to reduce their activity in the TC (100% for T2) and in the DC (66% and 100% for T1 and T2, respectively)

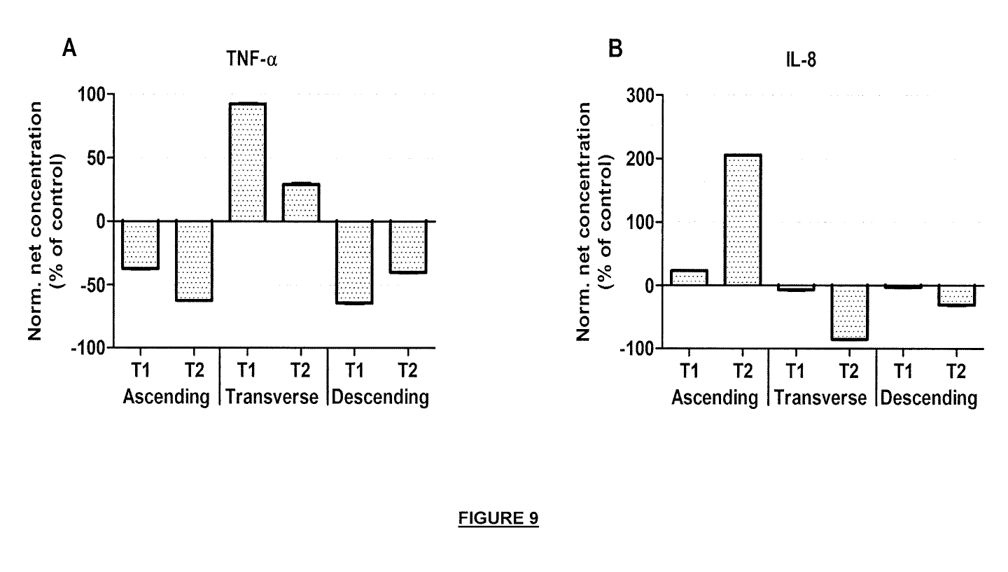

The net concentration o TNF-a and IL-8 in the different parts of the colon (AC, TC and DC)) of the SHIME treated with AG (cf. Example 1) was measured (FIG. 9).

TNF-α had a fluctuating trend in both SHIMEs. In fact, in the AC, AG was able to reduce TNF-α (FIG. 9 A) when compared to the control: −37% (T1) and −62% (T2) for AG. In the TC TNF-α secretion increased again: +25% (T1) and +23% (T2) for FOS; +92% (T1) and +29% (T2) for AG. Finally, in the DC, TNF-α levels were again inhibited: −64% (T1) and −40% (T2) for AG. In all cases, the activity of AG showed the stronger extent in terms of variation from the control.

Secretion of IL-8, after an initial increase observed in the AC, was inhibited by AG in the simulated transverse and descending colon compartments in the second week of treatment (−85% and −31% for TC and DC, respectively) (FIG. 9 B).

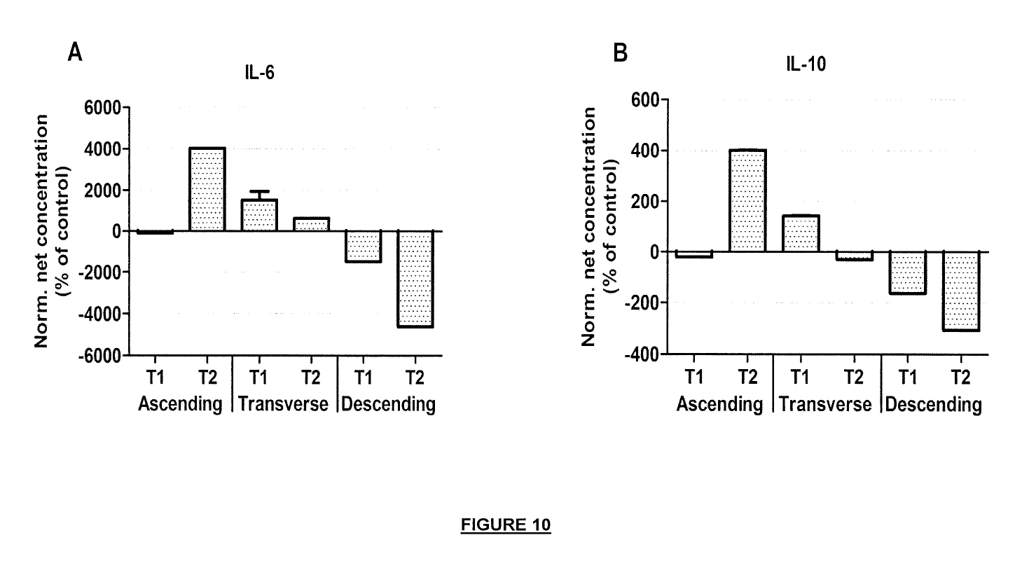

The net concentration of IL-6 and IL-10 in the different parts of the colon (AC, TC and DC)) of the SHIME treated with AG (cf. Example 1) was measured (FIG. 10).

AG was able to modulate IL-6 and IL-10 secretion (FIG. 10). More specifically, IL-6 (FIG. 10 A) secretion, after an initial increase in the AC, was gradually inhibited by AG, being clearly reduced in the DC when compared to the control, particularly in the second week of treatment. Finally, IL-10 (FIG. 10 B), a bona fide anti-inflammatory cytokine, was induced in the AC, and then its levels gradually decreased towards the distal colon

In general, the opposite trends in some cytokines production observed for AG in the proximal and in the distal colon are in agreement with the preferential fermentation of AG in the distal colon. In fact, the contact of the cells with unprocessed fibers in the AC is possibly having little or adverse effects, but with progressive fermentation, the metabolites produced by the bacteria are having positive effects on the intestinal mucosa.

### EXAMPLE 4

### Pro-Anti-Inflammatory Activity in the IBD-M-SHIME

The description of the different parameters is the same provided for the IBS-SHIME.

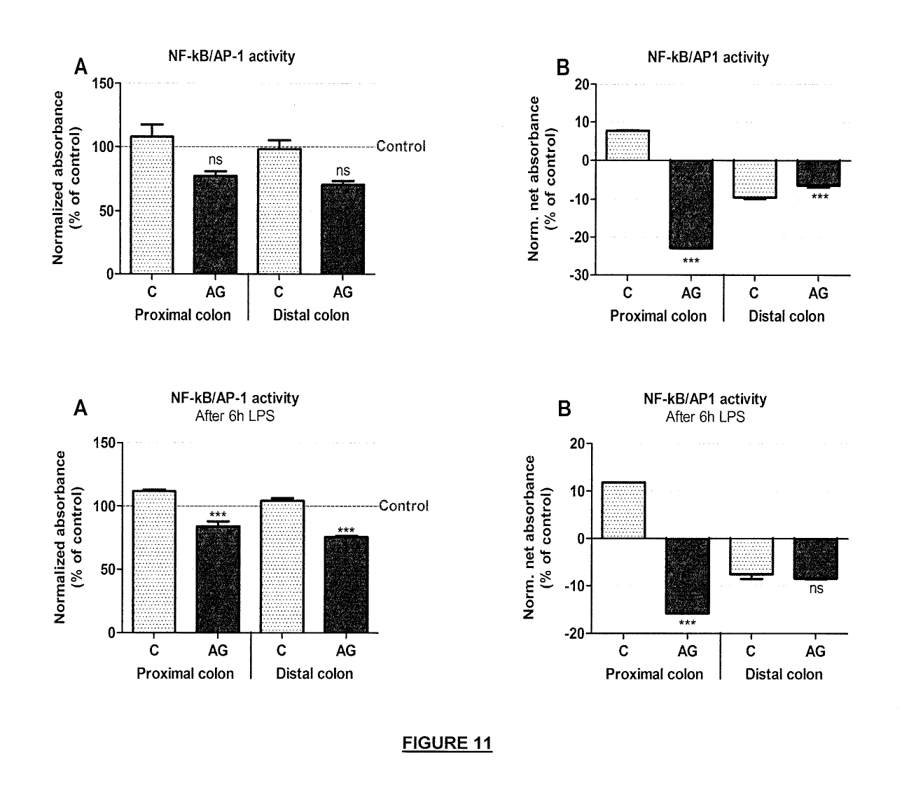

FIGS. 11-15 show the results of the effect of AG on pro-anti-inflammatory cytokines in the IBD-M-SHIME.

When focusing on FIG. 11, it is possible to observe that AG was able to decrease NF-κB/AP-1 activity of THP1-XBlue cells before (upper panels) and after LPS stimulation (lower panels) when compared to the control period (upper and lower A panels): −23% before and −16% after LPS addition in the proximal colon, and −29% before and −24% after LPS in the distal colon.

In general, NF-κB/AP-1 inhibition was more pronounced in the proximal colon when compared to the starch control after 4 days of fermentation (−31% for AG before LPS stimulation) (upper B panel). After LPS stimulation, AG was still able to decrease NF-κB/AP-1 activity when compared to the starch control after 4 days of fermentation (lower B panel).

In the distal colon, the inhibition of NF-κB/AP-1 activity was less pronounced when compared to the starch control, as the latter was also able to decrease the activity of the two transcription factors (upper and lower B panels).

AG was able to decrease NF-κB/AP-1 activity in both compartments.

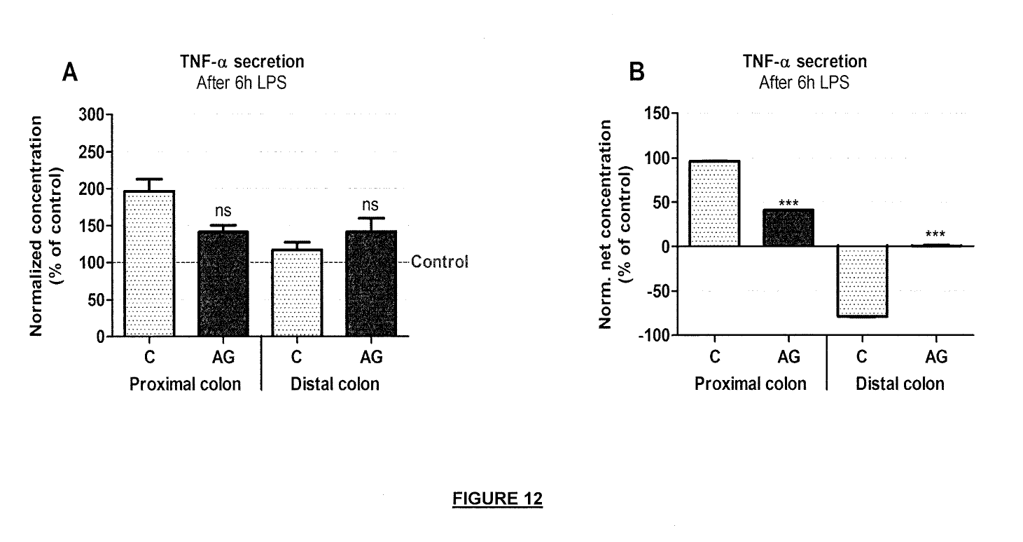

Concerning TNF-α levels, in general, all fibers (including starch) induced more TNF-α secretion as compared to the control period. However, when compared to the starch control (after 4 days of fermentation) AG showed lower levels of this cytokine in the proximal colon (−55% for AG; FIG. 12 panel A).

In the distal colon again, all fibers (including starch), induced higher TNF-α levels as compared to the control period (FIG. 12, panel A).

Considering the net results, AG did not change the secretion of TNF-α from one colon compartment to the other (FIG. 12, panel B).

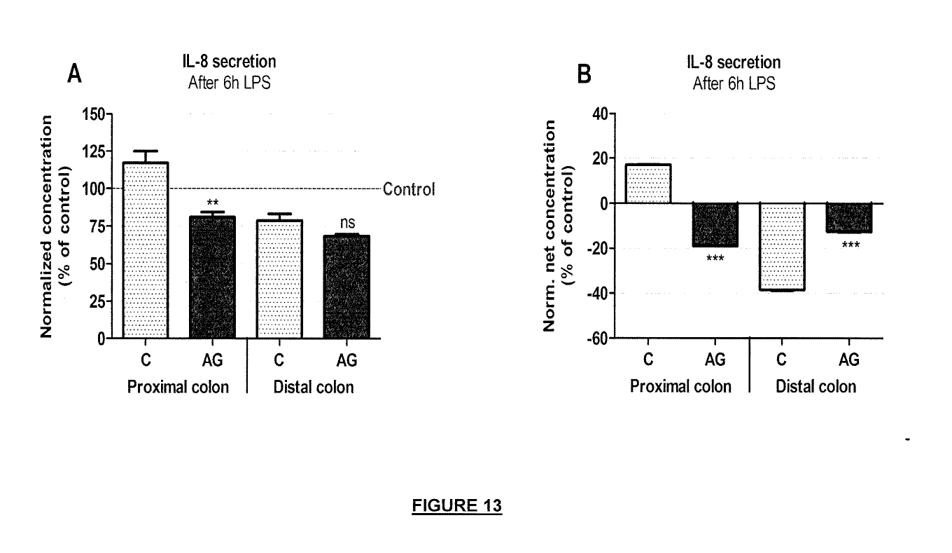

In contrast to TNF-α, IL-8 levels were reduced by AG after the control period in both colon compartments: −19% for AG in the proximal colon; −31% (AG) in the distal colon (FIG. 13 A).

However, when compared to the starch control after 4 days of fermentation, the difference was statistically significant only in the proximal colon: −36% for AG (FIG. 13, panel A).

Concerning the net results, AG was able to decrease this pro-inflammatory cytokine of 12% from the proximal to the distal colon (FIG. 13 B).

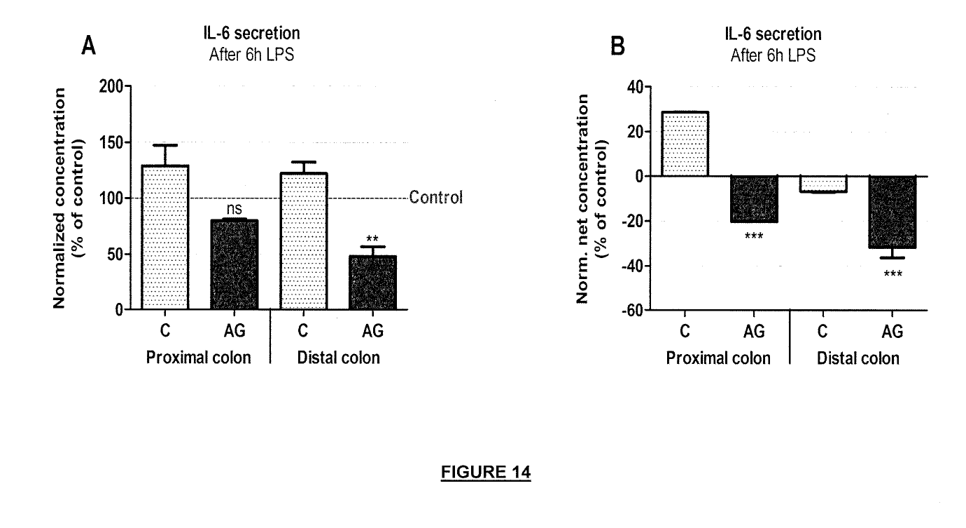

IL-6 levels follow a similar pattern as IL-8: when compared to the control period AG decreased IL-6 secretion of 20% in the proximal colon and 52% in the distal colon (FIG. 14 A).

The same specular trend could be observed also comparing the data to the starch control after 4 days of fermentation: in the proximal colon, (FIG. 14 A).

Concerning the net production (FIG. 14 B), AG showed an opposite trend: a 32%-decrease of IL-6 from the proximal to the distal colon.

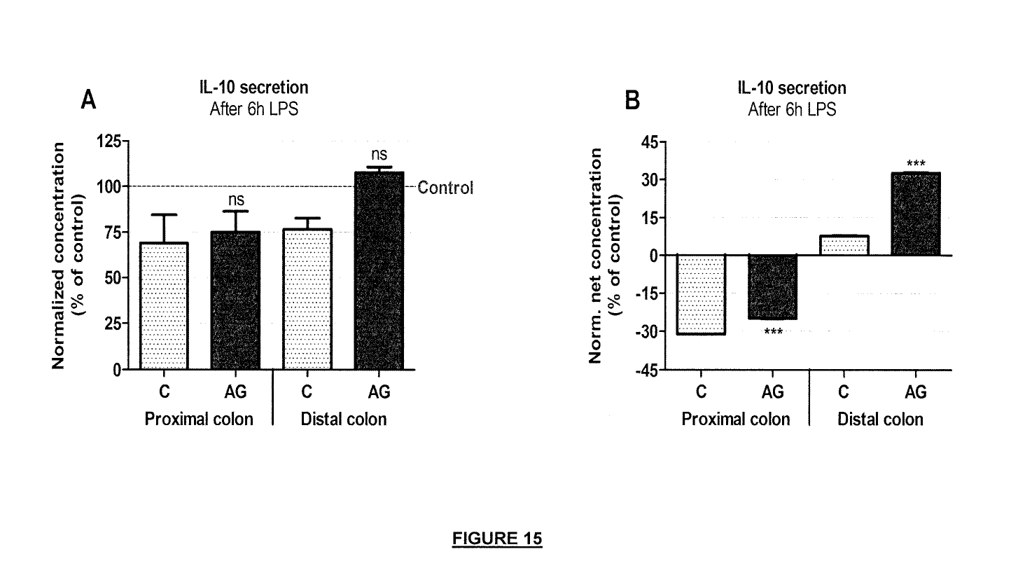

Finally, FIG. 15 shows the data related to IL-10 production, a bona fide anti-inflammatory cytokine. AG (+8% in the distal colon) is able to induce this cytokine levels when compared to the control period (FIG. 15 A).

When compared to the starch control (after 4 days of fermentation) AG induced a strong increase of IL-10 levels in the distal colon (+31%)—the main area of fermentation of this product—only a small increase was observed in the proximal colon (+6%).

As a consequence of these effects, when analyzing the net production of IL-10 (FIG. 15 B), AG induced an increase (+33%) of IL-10 levels from the proximal to the distal colon.

1. A method for improving gut impermeability comprising administering to a subject a sufficient amount of a composition comprising acacia gum (AG).

2. The method of claim 1 wherein the composition further comprises fructo-oligosaccharides (FOS).

3. The method of claim 2 wherein the composition comprises FOS that present in an amount of 1 to 50% percent of the weight of the composition.

4. The method of claim 1 wherein in the composition further comprises at least one of the following amino acids L-glutamine, non-fermentescible carbohydrates, fructo-oligosaccharides (FOS); and/or vitamin D, quercetin; turmeric, aloe vera, plantain, calendula; essential fatty acids; probiotics; minerals; and/or enzymes.

5. The method of claim 1 wherein the composition comprises from 1 to 60 g, preferably from 5 to 30 g of AG.

6. The method of claim 1 wherein the composition is in solid form such as tablets, bakery products, confectionary, capsules, powder sachets and boxes or in liquid form.

7. The method of claim 1 wherein the composition is administrated one to three times a day continuously during the year, during a period of 1 to 25 weeks.

8. The method of claim 1 wherein the subject has at least one of the following conditions: abdominal pain, chronic or not, insomnia, bloating, flatulence, shortness of breath, gluten intolerance, malnutrition, muscle cramps, multiple chemical sensitivities, muscle pain, mood swings, poor exercise tolerance, poor immunity, poor memory, recurrent skin rashes, brittle nails, hair loss, food allergies, constipation, diarrhea, liver dysfunction, brain fatigue, abdominal spasms, constant hunger pains, depleted appetite, Irritable Bowel Syndrome, chemotherapy, food allergies, acne, liver dysfunction or inflammation of the bowel or to treat anaphylactoid reactions, fevers of unknown origin, anxiety, hemorrhoids, heartburn, migraines, myofascial pain, mood swings, poor exercise tolerance, recurrent bladder infections, recurrent vaginal infections, swollen lympth glands, anal irritation, depression” sluggishness, celiac disease, multiple Sclerosis, autism, fibromyalgia, Chronic Fatigue Syndrome, eczema, dermatitis, ulcerative colitis, candidiasis, chronic hepatitis, asthma, cystic fibrosis, accelerated aging, endotoxemia, colon cancer, Crohn's disease, giardia, arthritis, pancreatic dysfunction, Hives, alcoholism, anyklosing spondylitis, inflammatory bowel disease, liver dysfunction, psoriasis, schizophrenia, alcoholism.
